(19) United States
(12) Patent Application Publication
(10) Pub. No.: US 2007/0233187 A1
(43) Pub. Date: Oct. 4, 2007

(54) Self-closing medical staple

(75) Inventor: Richard D. Lobello, Johnston, RI (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/397,269

(22) Filed: Apr. 4, 2006

(51) Int. Cl. A61B 17/08

(52) U.S. Cl. 606/219

(57) ABSTRACT

A self-closing medical staple including a resilient body having an axis and a longitudinal opening, and at least two opposed longitudinal tines spaced about the axis. Each tine is partially enclosed by the body and has a distal end exposed distally of the body. The staple has a closed configuration wherein at least the distal ends of the tines are disposed adjacent to or contacting each other and an open configuration wherein at least the distal ends of the tines are radially separated from each other. A wound closure system includes a mandrel with a flared portion for sliding movement within the longitudinal opening to transform the staple between the open and closed configurations. The system includes a pusher for advancing the staple into tissue surrounding a wound. A method of closing a wound with the self-closing staple is also disclosed.

### FIELD OF THE DISCLOSURE

The present disclosure is generally directed to a medical staple, and is more particularly directed to a self-closing medical staple which may be used for closing a vascular wound. A staple, a stapler, and a method in accordance with the invention may be used for blind closure of a puncture arteriotomy.

### BACKGROUND OF THE DISCLOSURE

Intravascular catheters and catheterization procedures are used for a number of diagnostic and therapeutic applications, and access to the arterial system typically involves insertions into the femoral artery. After a diagnostic or therapeutic catheterization, the arterial wall puncture formed for, or by the catheter must be closed. The size of the puncture opening in the artery corresponds to the size of the catheter or percutaneous introducer sheath used, which devices may typically range in diameter from 5 French (1.67 mm) for a diagnostic procedure to 6-10 French (2.00 mm-3.33 mm) for a therapeutic procedure. Traditionally, compression has been applied to the puncture site for at least 30-45 minutes for the wound to close naturally after removal of the catheter. Patients are required to remain decumbent, essentially motionless and often with a heavy sandbag placed on their upper leg, for several hours to ensure that the bleeding has stopped. This traditional method of arteriotomy closure following femoral artery access has many inadequacies. When a coronary artery blockage is opened during an interventional catheterization procedure such as angioplasty and/or stent deployment, the patient quickly feels better and they often have more energy than they have had in years, but they must lie down uncomfortably for several hours. The weight of the sandbag on the femoral artery may cause the lower leg to tingle or go numb. The recovery time from the medical procedure may be as little as half of an hour, but the recovery time from the wound can exceed 24 hours. This makes wound site management the longer critical care item. The longer the recovery time, the more expensive the procedure becomes, the greater the patient discomfort, and the greater the risk of complications. Other approaches to arteriotomy closure include a compression clamp device, a thrombotic or collagen plug, and/or suturing devices.

Medical stapling systems have been proposed to resolve some of the concerns associated with arteriotomy closure after vascular catheterization procedures. Many medical stapling instruments and systems involve the use of a staple that is forced against an anvil in order to bend the ends of the staple into a secure closed configuration. Such systems, however, often require the anvil to be positioned within the interior of the lumen. After the staple has been secured, the anvil must be extracted from the lumen. The need to extract the anvil after the staple has been secured may require that the access wound be less than completely closed. While the wound may be closed to a greater degree than without the use of such a closure system, additional procedures may still be necessary to achieve complete hemostasis.

Medical stapling systems wherein the anvil remains external to the blood vessel are also known, but such systems may be difficult and expensive to make precisely in the small dimensions that are advantageous for blind closure of femoral arteriotomies. Self-closing medical staples are also known. Such devices rely on elastic or super-elastic properties of the metal used in their construction to transform the staple from an open configuration to a closed configuration. Self-closing staples do not require a deforming anvil to deliver the staple, but the associated delivery system must hold the staple in the open configuration while staple tips are inserted into the wound tissue. Such all-metal, self-closing, medical staples and their associated delivery systems may also be difficult and expensive to make precisely in the small dimensions required. There is therefore a need for an improved medical stapling system that is simple and inexpensive to manufacture.

### SUMMARY OF THE DISCLOSURE

According to one aspect of the disclosure, a medical staple is disclosed that includes two or more tines that are at least partially enclosed by an elastically deformable, or resilient body. The tines are movable with respect to each other between a closed configuration and a open configuration through elastic deformation of the resilient body.

According to another aspect of the disclosure, a method is provided for closing a wound. The method includes providing a staple including an elastically deformable, or resilient body that at least partially encloses the two or more tines. The method may further include moving the tines to an open, spaced-apart configuration and engaging tissue surrounding the wound with the tines. The tines may be urged toward one another by the resilient body into a closed configuration that closes the wound.

Still another aspect of the disclosure is a wound closure system. The wound closure system includes a staple having two or more tines and an elastically deformable, or resilient body which at least partially encloses at least a portion of each tine. The wound closure system may also include a flared mandrel capable of elastically deforming the resilient body of the staple to move the tines between an open configuration and a closed configuration.

### BRIEF DESCRIPTION OF THE DRAWINGS

The foregoing and other features and advantages of the disclosure will be apparent from the following description of the disclosure as illustrated in the accompanying drawings. The accompanying drawings, which are incorporated herein and form a part of the specification, further serve to explain the principles of the disclosure and to enable a person skilled in the pertinent art to make and use the disclosure. The drawings are not to scale.

FIG. 1 is a rear, or proximal perspective view of an embodiment of a wound closure system consistent with the present disclosure;

FIG. 2 is a front, or distal perspective view of an embodiment of a wound closure system consistent with the present disclosure;

FIG. 3 is a side view of an embodiment of a wound closure system consistent with the present disclosure, a staple being shown in longitudinal section;

FIG. 4 is a perspective view of a tine which may be used in connection with a staple consistent with the present disclosure;

FIG. 5 is a front, or distal view of an embodiment of a staple consistent with the present disclosure;

FIG. 6 illustrates a side view of a wound closure system of the present disclosure with a staple of the disclosure in a closed configuration;

FIG. 7 illustrates a cutaway side view of a wound closure system of the present disclosure with a staple of the disclosure in an open configuration;

FIG. 8 illustrates a cutaway side view of a wound closure system of the present disclosure with a staple of the disclosure engaging tissue adjacent a wound in a patient's blood vessel;

FIG. 9 illustrates a cutaway side view of a wound closure system of the present disclosure with a staple of the disclosure engaged with tissue adjacent a wound in a patient's blood vessel, the staple shown in transition from an open configuration to a closed configuration;

FIG. 10 illustrates a cutaway side view of a wound closure system of the present disclosure with a staple of the disclosure engaged with tissue adjacent a wound in a patient's blood vessel, the staple shown in a closed configuration; and

FIG. 11 illustrates a cutaway side view of a wound closure system of the present disclosure with a staple of the disclosure engaged with tissue adjacent a wound in a patient's blood vessel, the staple shown in a closed configuration during disengagement of a mandrel.

### DETAILED DESCRIPTION OF THE DISCLOSURE

Specific embodiments of the present disclosure are now described with reference to the figures. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

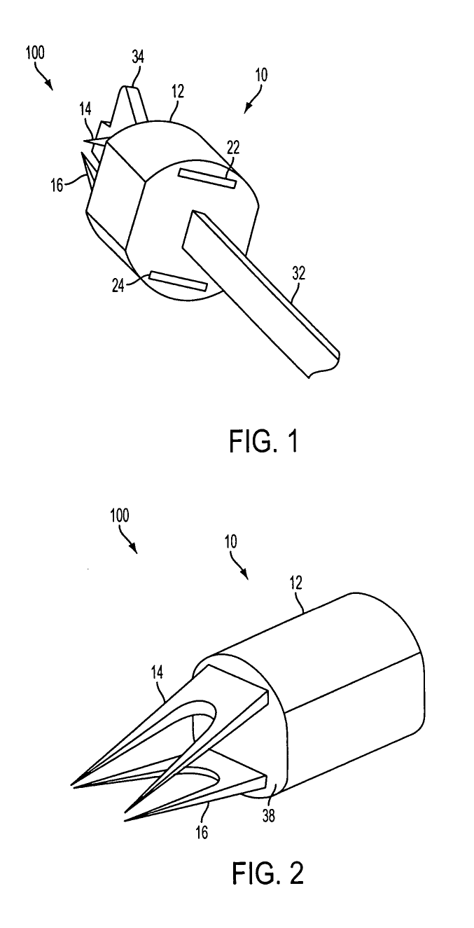

Referring to FIGS. 1 and 2, a medical stapling system 100 is shown. A staple 10 includes an elastically deformable or resilient body 12 having two or more distinct longitudinal tines 14, 16 enclosed by and extending distally from body 12. Staple 10 may generally be configured for closing a wound, such as a puncture, incision, etc. Staple 10 may be particularly suited for closing a wound formed in a vessel, such as may be created in a blood vessel during the course of a catheterization procedure. Staple 10 may provide wound closure by engaging tines 14, 16 with tissue adjacent the wound, e.g., tissue on opposite sides of a wound, while tines 14, 16 are in an open configuration, i.e., while at least a portion of tines 14, 16 are spaced or spread apart from one another. With tissue surrounding the wound engaged by tines 14, 16, staple 10 may be urged toward a closed configuration by moving at least a portion of tines 14, 16 toward one another.

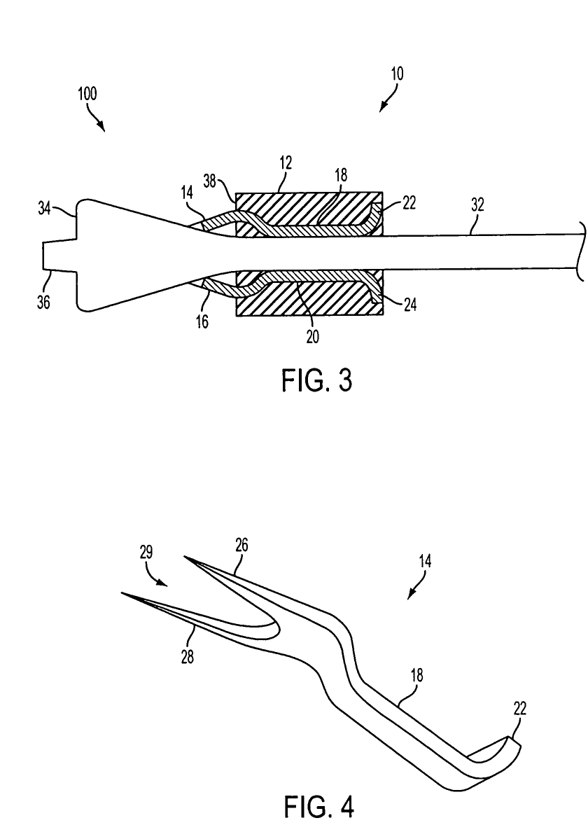

Tines 14, 16 of staple 10 may be moved between the closed and open configurations by elastically deforming body 12. In this regard, body 12 may be formed from an elastically deformable, or resilient material. As shown in the cross-sectional view of FIG. 3, each of tines 14, 16 has at least a portion that may be encapsulated or enclosed by body 12. As noted, movement of tines 14, 16 between the closed and open configurations may involve applying a deforming stress to resilient body 12. For example, in a relaxed state, body 12 may bias tines 14, 16 toward the closed configuration. To move tines 14, 16 to the open configuration, resilient body 12 may be elastically deformed by moving at least a portion of tines 14, 16 away from one another.

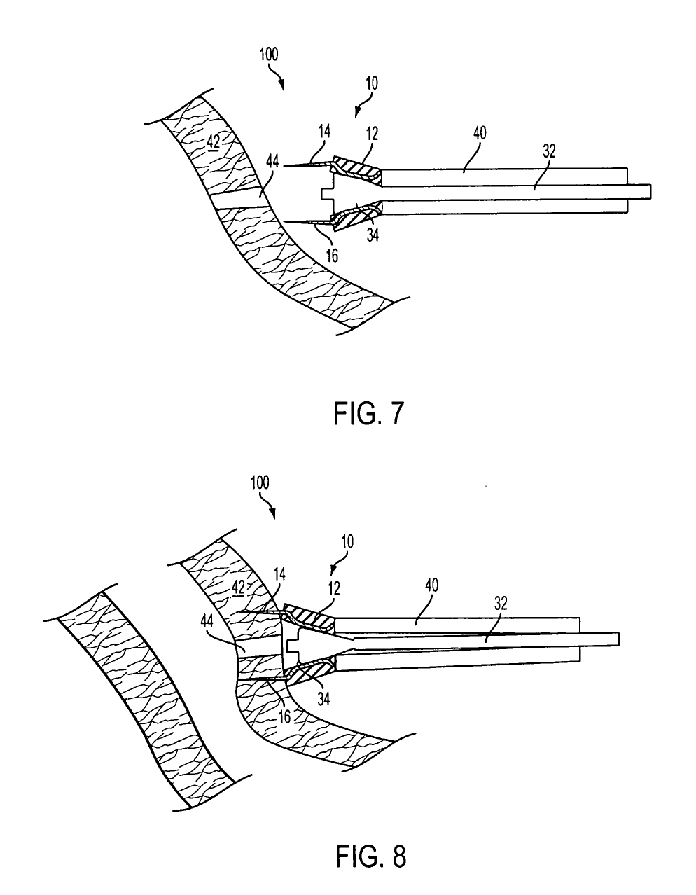

Staple 10 is shown in the closed configuration in FIGS. 1 and 2. In the closed configuration the distal ends of tines 14, 16, i.e., the ends of tines 14, 16 away from resilient body 12, may be near one another, and may be parallel (not shown) or generally angled toward one another. In the open configuration (see FIG. 7), at least a portion of tines 14, 16 may be moved away from one another. For example, the distal ends of tines 14, 16 may be angled away from one another. In other embodiments, the tines may be moved to an open configuration by spreading the tines laterally away from one another, without necessarily changing the angular relationship there between. Various other suitable embodiments may also be achieved in which at least a portion of the tines are moved away from one another by the elastic deformation of the resilient body to enable the tines to engage tissue surrounding or adjacent a wound. At least a portion of the tines may be moved toward one another by the elastic recovery of the resilient body to urge together wound tissue engaged by the tines.

Resilient body 12 may comprise a non-metallic, biocompatible, natural or synthetic rubber such as butadiene/acrylonitrile copolymers, copolyesters, ethylene vinylacetate (EVA) polymers, ethylene/acrylic copolymers, ethylene/propylene copolymers, fluorosilicone, latex, polyalkylacrylate polymers, polybutadiene, polybutylene, polyethylene, polyisobutylene, polyisoprene, polyurethane, silicone, styrenebutadiene copolymers, styrene-ethylene/butylene-styrene, thermoset elastomer, thermoplastic elastomer and combinations of the above. Resilient body 12 may be formed by various methods including casting, compression molding, liquid injection molding, reaction injection molding (RIM), resin transfer molding (RTM), thermoplastic injection molding and two-shot molding. Resilient body 12 may be molded of soft foam, solid elastic material, or a combination thereof.

Resilient body 12 may be insert molded or over-molded directly around and encapsulating at least portions of tines 14, 16. Alternatively, body 12 may be formed with cavities for subsequently receiving tines 14, 16. Such cavities in body 12 may be through-holes, open-sided indentations or blind pockets and may be formed in sizes and shapes to closely match tines 14, 16. Optionally, the cavities may be formed undersized or otherwise sized and shaped to require a degree of distortion in body 12 to force tines 14, 16 into or out of the cavities therein. Tines 14, 16 may be retained in body 12 by various means such as inherent mechanical friction, a chemical bond between the materials of tines 14, 16 and body 12, a biocompatible adhesive, or by use of anchor elements, such as protrusions or indentations on tines 14, 16 to mechanically engage the tines with the material of body 12.

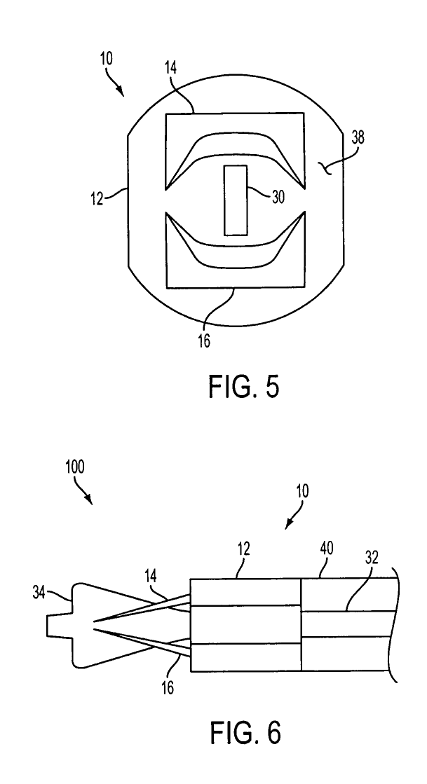

As further illustrated in cross-section in FIG. 3, a mandrel 32 extends through staple 10 and comprises a stapler as part of staple delivery system 100. Mandrel 32 may be slidably movable through an opening 30, as described in further detail below with respect to FIG. 5. Mandrel 32 may include a distally diverging or flared portion 34. In the illustrated embodiment, which shows only two tines 14 and 16, mandrel 32 and flared mandrel portion 34 can be flat, or rectangular in cross section to be disposed transversely between opposing tines 14 and 16. In optional embodiments (not shown) having three or more tines, the mandrel and/or the flared mandrel portion may have a transverse cross section that is polygonal or circular, which could result in a conical flared mandrel portion.

Mandrel 32 may also include a locating tab 36 at the distal end thereof. In various embodiments, locating tab 36 may be used to align mandrel 32, as well as staple 10 carried on mandrel 32, with a wound. For example, locating tab 36 may, in some embodiments, be substantially longer than shown to facilitate being received in a wound during a stapling procedure. In such an embodiment, tab 36 may include further positioning elements, such as temporarily inflatable, expandable or deformable anchoring members (not shown) for deployment beyond or in abutment with the luminal (interior) surface of the vessel wall adjacent the wound.

As also shown in FIG. 3, tines 14, 16 may extend through at least a portion of resilient body 12 in a generally parallel orientation relative to one another, although other configurations may suitably be employed. The central portions 18, 20 of tines 14, 16 extending through body 12 may be completely enclosed by the body 12, or may be partially exposed, e.g. along opening 30. As shown, proximal ends 22, 24 of tines 14, 16 respectively, may at least partially diverge from the generally parallel arrangement of the tines. Such divergent tine proximal ends 22, 24 may act as anchors, providing resistance to tines 14, 16 being pulled from body 12, as described above.

In the embodiment shown in FIG. 3, tine proximal ends 22, 24 are not fully encapsulated by the material of resilient body 12. Instead, at least the proximally-facing surfaces of tine proximal ends 22, 24 are exposed at the proximal end of body 12 to provide rigid surfaces for direct abutment with the distal end of staple pusher 40 (see FIG. 6). As described more fully below, pusher 40 is capable of holding staple 10 while mandrel 32 is pulled proximally through staple 10. Pusher 40 is also capable of pushing staple 10 distally such that tines 14, 16 pierce tissue surrounding a wound. In alternative embodiments (not shown), tine proximal ends 22, 24 may be embedded below the proximal surface of body 12, or tine proximal ends 22, 24 may extend proximally some distance from the proximal end of body 12.

With additional reference to FIG. 4, tine 14 may include one or more tissue-engaging prongs 26, 28 at the distal end 29 thereof. Tissue-engaging prongs 26, 28 may be capable of at least partially piercing tissue adjacent a wound. The sizes and shapes of the tines of staple 10 may be identical, or chiral (mirror-image) or dissimilar. The tines are arranged radially around a center axis of staple 10 such that the one or more tissue-engaging prongs of each tine are disposed in opposed mating relationship with the one or more tissue-engaging prongs of the generally opposed tine(s). The tines may be generally angularly equispaced about the center axis of staple 10. In another embodiment (not shown), the one or more tissue-engaging prongs of each tine are disposed in staggered relationship with the one or more tissue-engaging prongs of the generally opposed tine(s) such that opposing prongs may be interposed with each other in the closed configuration. In the example shown, tines 14 and 16 are identical in size and shape and are disposed opposite each other such that the tissue-engaging prongs are in a mutually contacting, or at least a mutually adjacent mating relationship when staple 10 is in the closed configuration.

Tines 14, 16 may be formed from a variety of suitable biocompatible materials. Because resilient body 12 may provide a portion or substantially all of the self-closing function of staple 10, tines 14, 16 do not need to be sufficiently ductile for forming about an anvil, nor do tines 14, 16 need to be sufficiently elastic to be resiliently deformed during deployment. Thus, the variety of suitable materials and methods of making tines 14, 16 is relatively broader than the materials required for known medical staple designs. Suitable materials for tines 14, 16 may include ceramic or metallic materials, such as stainless steel, tantalum, titanium. Magnesium or an alloy thereof has the potential advantage of being bioabsorbable.

Tines 14, 16 may also be formed from various filled or unfilled rigid polymers such as liquid crystal polymer (LCP), polyamide, polycarbonate, poly-etheretherketone (PEEK), polysulfone, polyvinylidene fluoride (PVDF), and may include bioabsorbable or biodegradable polymeric materials such as polycaprolactone, poly(glycolide) (PGA), poly(L-lactide) (PLLA) and poly(D,L-lactide) (PLA). In an example of a method of making a staple embodiment of disclosure, tines 14, 16 may formed of a rigid thermoplastic in a first injection mold. Molded tines 14, 16 are then transferred to a second mold for over-molding portions of the tines with an elastic material to form resilient body 12, thus creating staple 10. Such a combination of two molding steps is sometimes referred to as double-shot molding.

Turning to FIG. 5, staple 10 may include opening 30 extending through at least a portion of body 12. Opening 30 may extend generally axially through body 12. As shown in FIG. 3, central portions 18, 20 of tines 14, 16 may define, or be adjacent to at least a portion of opening 30. Opening 30 may be sized and shaped to be capable of receiving mandrel 32 at least partially there through.

The distal face 38 of resilient body 12 adjacent to the extending tines 14, 16, may be employed as a pledget surface. That is, when the staple is deployed in a wound site, face 38 may be positioned in contact with, or adjacent to, the tissue surrounding the wound, and may overlie at least a portion of the wound. Face 38 may apply a force to the tissue, which may have the effect of a compress to at least partially stem the flow of bodily fluid from the wound. Even if face 38 does not contact or compress against the wound, the close proximity of face 38 may resist the flow of fluid. The close proximity of face 38 to the wound may also assist clotting and facilitate the natural cessation of flow.

In a further embodiment, the pledget may be provided as a discrete component (not shown) provided on, or adjacent to distal face 38 of resilient body 12. For example, a pledget may include a disc, fabric, film, membrane, pad, sheet etc., which may be disposed on, or adjacent to distal face 38. The discrete pledget may be formed of a biocompatible and/or bioabsorbable or biodegradable material such as the materials mentioned above for forming resilient body 12.

Whether a discrete component or an integral part of body 12, the pledget may be capable of absorbing bodily fluid or the pledget surface may be used to deliver one or more pharmaceutical agents, e.g., an anti-microbial agent, an anti-thrombotic agent, or a pro-thrombotic agent. Such pharmaceutical agents may be coated on the pledget surface, absorbed in the pledget, or be included in a drug-eluting polymeric composition of the pledget.

FIGS. 6 through 11 illustrate an embodiment of medical stapling system 100 and a method of using system 100 for at least partially closing a wound 44, which, in this example, is a puncture in a vessel wall. As shown in FIG. 6, mandrel 32 extends through an opening in resilient staple body 12. Flared portion 34 of mandrel 32 is disposed distally adjacent to tines 14, 16 of staple 10 to allow staple 10 to be in a first, relaxed, closed configuration with opposed tissue-engaging prongs in contact with, or adjacent to each other. Pusher 40 has a distal end for abutting and pushing against a proximal end of staple 10. Pusher 40 will be described in further detail below.

As shown in FIG. 7, staple 10 has been transformed from the closed configuration shown in FIG. 6 to a second, open configuration in which at least the distal ends 29 of tines 14, 16 are radially or transversely separated from one another. Tines 14, 16 are moved to the open configuration by moving mandrel 32 proximally relative to staple 10. Proximal movement of mandrel 32 draws flared portion 34 of mandrel 32 at least partially into opening 30 to elastically deform body 12 of staple 10. For example, flared portion 34 of mandrel 32 causes at least a portion of resilient body 12 to expand or stretch to induce or accommodate the increased separation of tines 14, 16. In the embodiment shown, flared mandrel portion 34 impinges on at least portions of tines 14, 16, e.g., a portion of each tine that may be at least partially enclosed by resilient body 12, and wedges tines 14, 16 apart. At least a portion of one tine may be moved apart or away from the other tine as body 12 is elastically deformed.

FIG. 7 also illustrates medical stapling system 100 positioned adjacent to, and aligned with wound 44 through the wall of a body vessel. In an example, the vessel may be an artery, which is typically reached during a catheterization procedure by creating a puncture wound that extends not only through the arterial wall, but also through the patient's skin and superficial and deep layers of fascia, which layers of tissue are not shown for clarity. Embodiments of the disclosure may be used to close an arteriotomy blindly, i.e. without opening or further separating the overlying tissue layers to provide direct visualization of the wound through the arterial wall. Guidance during such blind wound closure procedures may be aided by locating tab 36 of mandrel 32, as described above.

Flared mandrel portion 34 may be translated proximally relative to staple 10 by supporting staple 10 with pusher 40 and withdrawing mandrel 32 proximally relative to staple 10 and pusher 40. In this manner, pusher 40 may provide a counter force against proximal movement of staple 10 as mandrel 32 is withdrawn there through. In a related manner, pusher 40 may advance staple 10 distally along mandrel 32. The steps of proximally withdrawing mandrel 32 and distally advancing pusher 40 may be performed simultaneously, sequentially, or in any combination of such motions to move staple 10 relative to mandrel 32 for elastically deforming body 12 about flared mandrel portion 34 and for moving tines 14, 16 toward the open configuration. Accordingly, moving mandrel 32 proximally relative to staple 10 and moving staple 10 distally relative to mandrel 32 should be construed as moving mandrel 32 and staple 10 relative to one another, rather than moving one component while maintaining the other stationary. A control handle (not shown) may be incorporated in wound closure system 100 for external or proximal manipulation of mandrel 32 and pusher 40 as described above for deployment of staple 10.

Referring next to FIG. 8, with staple 10 in an open configuration, as shown in FIG. 7, tines 14, 16 may be engaged with tissue 42, e.g., a vessel wall, adjacent wound 44. For example, staple 10, along with mandrel 32 and pusher 40 may be moved in unison toward wound 44, causing the prongs of tines 14, 16 to at least partially pierce tissue 42. In an alternative manner, pusher 40 may be moved distally relative to mandrel 32, urging staple 10 distally relative to mandrel 32 and advancing staple 10 to engage tissue 42. Staple 10 may be advanced against tissue 42 until a tissue stop is reached. In the embodiment shown in FIG. 8, resilient body 12, and more particularly the distal face of body 12 acts as a tissue stop to limit the depth to which tines 14, 16 are inserted into the vessel wall. In this embodiment, the penetration depth of tines 14, 16 is determined by the extent to which the tines are exposed, or uncovered, distally of resilient body 12. Alternatively, in the embodiment shown in FIG. 2, each of tines 14, 16 may be forked into two tissue-piercing prongs 26, 28, and the crotch formed between the prongs may act as a tissue stop. Other types of tissue stops may be provided on staple 10, such as a discrete pledget of a selected thickness, or one or more protrusions (not shown) extending laterally from one or more prongs.

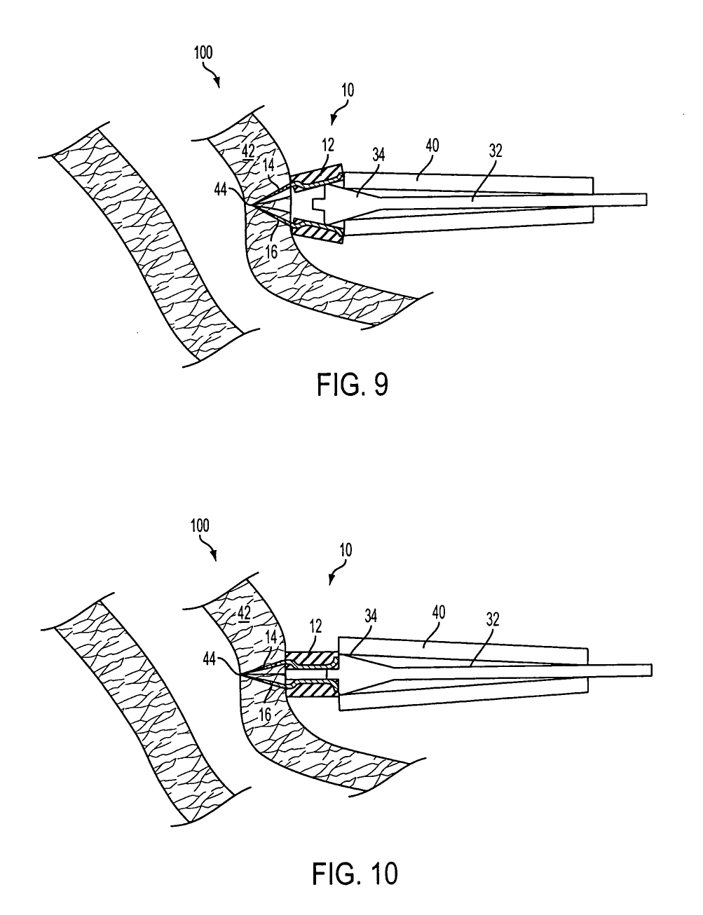

With additional reference to FIG. 9, mandrel 32 may continue to be withdrawn proximally relative to staple 10, drawing flared portion 34 of mandrel 32 through resilient body 12 of staple 10. As flared portion 34 of mandrel 32 moves through body 12, body 12 may elastically recover such that tines 14, 16 revert toward the closed configuration. For example, while flared portion 34 of mandrel 32 is disposed in the proximal region of body 12, the distal region of body 12 may begin elastically recovering, causing the distal ends 29 of tines 14, 16 to move toward one another. As tines 14, 16 revert toward the closed configuration, tissue-engaging prongs 26, 28 of staple 10 may draw together tissue 42, at least partially closing or reducing an open dimension of wound 44.

Pusher 40 may comprise an elongate tube that slidably receives mandrel 32 and may have a substantially orthogonal distal end for abutment against the proximal end of staple 10. Additionally, as flared portion 34 of mandrel 32 moves proximally through, and begins to exit staple 10, at least a distal region of pusher 40 may expand radially or laterally to accommodate or receive flared mandrel portion 34. Such expansion of pusher 40 may include resilient or non-resilient uniform radial expansion or lateral expansion, or longitudinal splitting or tearing along one or more slits, seams or tear lines.

In an alternative embodiment, staple pusher 40 may include two or more longitudinal pusher members for abutment against the proximal end of staple 10. The longitudinal pusher members may at least partially separate laterally to receive flared mandrel portion 34 there between. The longitudinal pusher members may be joined together at their proximal ends, at their distal ends, at multiple locations or continuously along their lengths. The number of longitudinal pusher members may correspond to the number of tines and the longitudinal pusher members may be aligned or registered to push directly on the proximal ends of the tines. Other pusher embodiments may also suitably accommodate mandrel 32 as it is moved through staple 10. As another example, a pusher tube embodiment may be provided with longitudinal slots or notches at the distal end for slidably receiving flared mandrel portion 34 without necessarily requiring radial or lateral expansion of the pusher tube.

As mandrel 32 is moved farther proximally relative to staple 10, reduction or elimination of a deforming stress on resilient body 12 may allow body 12 to elastically revert farther towards the closed configuration. The elastic recovery of body 12 may continue to urge at least a portion of tines 14, 16 toward one another, as shown in FIG. 10. For example, resilient body 12 may bias at least a portion, e.g. distal ends 29 of tines 14, 16 toward one another, i.e., toward the closed configuration. FIG. 9 shows distal ends 29 of tines 14, 16 having returned to a partially closed configuration wherein ends 29 are relatively adjacent one another, while proximal portions of tines 14, 16 remain separated by flared portion 34 of mandrel 32. FIG. 10 shows both proximal and distal ends 29 of tines 14, 16 have reverted to the closed configuration. As body 12 continues to elastically recover, tissue 42 adjacent wound 44 may continue to be drawn together, at least partially closing wound 44.

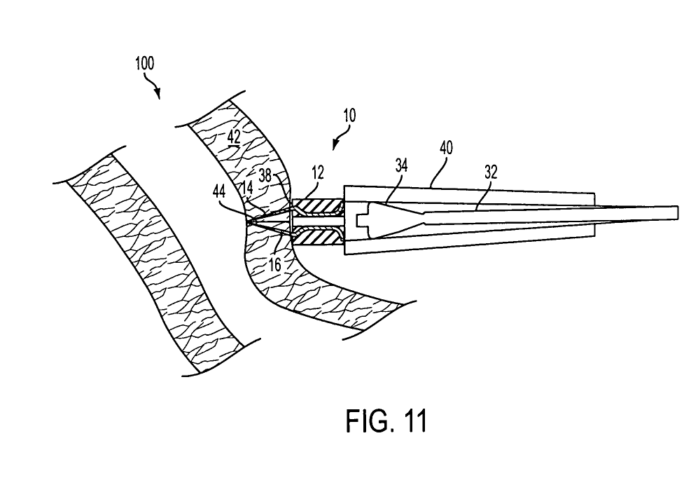

As mandrel 32 is completely withdrawn from staple 10, as shown in FIG. 11, resilient body 12 of staple 10 may generally recover to a pre-deformed configuration. As used in any embodiment herein, elastic deformation of body 12 may include both plastic and elastic deformation. Correspondingly, while body 12 of staple 10 may elastically recover to move at least a portion of tines 14, 16 together, and may bias the staple toward the closed configuration, some degree of plastic deformation may remain. Accordingly, in an elastically recovered condition, staple 10 need not fully recover an initial condition, i.e., the pre-deformed configuration. Additionally, engagement between staple 10 and a sufficient span of tissue 42 may maintain staple 10 in an at least partially deformed condition, even after mandrel 32 has been withdrawn from staple 10 and/or after wound 44 has been completely closed. As further shown, pledget surface 38 of staple 10 may be approximated to wound 44. Once mandrel 32 has been completely withdrawn from staple 10, or as mandrel 32 is being withdrawn from staple 10, mandrel 32 and pusher 40 may be moved away from wound 44.

In summary, the staple illustrated and described herein generally includes a resilient body having at least two distinct tines that are at least partially enclosed by the resilient body. The resilient body may be elastically deformed, e.g., by stretching, expanding, etc., to spread at least a portion of the tines apart or away from one another. A mandrel having a flared portion has been described for elastically deforming the resilient body for moving the tines from a closed configuration to an open configuration. The tines may be engaged with tissue at least partially surrounding or defining a wound. The resilient body may then be allowed to at least partially elastically recover toward a closed configuration, drawing at least a portion of the tines toward one another. The tines may be biased toward the closed configuration by the resilient body. Engagement between the tines and tissue adjacent the wound may at least partially draw tissue adjacent the wound together to at least partially close the wound.

The principles associated with the described staple are susceptible to modification and application in numerous alternative embodiments that are considered to lie within the scope of this disclosure. For example, a staple may be provided having a resilient body and at least two tines. A fulcrum (not shown) may be provided between the tines adjacent a distal region of the staple. A proximal region of the tines may be moved toward one another to elastically deform the resilient body and to move the distal portions of the tines to an open configuration.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Other modifications, variations, and alternatives are also possible. Accordingly, the claims are intended to cover all such equivalents.

What is claimed is:

1. A self-closing medical staple comprising:
a non-metallic resilient body having an axis and a longitudinal opening; and
at least two opposed longitudinal tines spaced about the axis, each tine partially enclosed by the body and having a distal end exposed distally of the resilient body;
the staple having a closed configuration wherein at least the distal ends of the tines are disposed adjacent to or contacting each other and an open configuration wherein at least the distal ends of the tines are radially separated from each other;
wherein the staple is transformable from the closed configuration to the open configuration via deformation of the resilient body.

2. A staple according to claim 1, wherein the two or more tines are transversely opposed to each other.

3. A staple according to claim 1, wherein the two or more tines are radially opposed to each other.

4. A staple according to claim 1, wherein the tines are biased toward the closed configuration by the resilient body.

5. A staple according to claim 1, wherein the resilient body comprises a natural or synthetic rubber.

6. A staple according to claim 1, wherein the resilient body is molded around at least a portion of the tines.

7. A staple according to claim 1, wherein the longitudinal opening is capable of receiving a mandrel there through.

8. A staple according to claim 1, wherein each of the tines comprises at least one tissue-piercing prong.

9. A staple according to claim 1, wherein the resilient body comprises a pledget portion capable of contacting tissue adjacent a wound.

10. A wound closure system comprising:
a self-closing staple comprising:
a resilient body having an axis and a longitudinal opening;
at least two opposed longitudinal tines spaced about the axis, each tine partially enclosed by the body and having a distal end exposed distally of the resilient body;
the staple having a closed configuration wherein at least the distal ends of the tines are disposed adjacent to or contacting each other and an open configuration wherein at least the distal ends of the tines are radially separated from each other; and
a stapler comprising a mandrel slidably positionable within the longitudinal opening, the mandrel being adapted for deforming the resilient body to transform the staple between the closed and open configurations.

11. A system according to claim 10, wherein the mandrel comprises a flared portion that is positionable within the opening for elastically deforming the resilient body.

12. A system according to claim 11, wherein the mandrel further comprises a locating tab disposed at a distal end of the mandrel.

13. A system according to claim 11, wherein the stapler further comprises a pusher being adapted to slidingly receive the mandrel there through, and having a distal end sized and shaped for abutting a proximal end of the staple.

14. A system according to claim 13, wherein the pusher comprises two or more longitudinal pusher members for abutment against the proximal end of the staple.

15. A system according to claim 13, wherein the pusher is an elongate tube.

16. A system according to claim 15, wherein at least a distal region of the elongate tube is adapted to expand radially or laterally to receive the flared mandrel portion.

17. A system according to claim 16, wherein the distal region of the elongate tube comprises longitudinal slits, seams or tear lines.

18. A method for closing a wound comprising:
providing a self-closing staple comprising a resilient body having an axis, a longitudinal opening and at least two opposed longitudinal tines spaced about the axis, each tine partially enclosed by the body;
moving a portion of a mandrel into the body opening to move at least a distal portion of each tine to a spaced apart configuration;
engaging tissue with the tines, the tissue at least partially surrounding the wound; and
moving the portion of a mandrel within the body opening to allow at least a portion of the tines to revert towards one another to at least partially close the wound.

19. A method according to claim 18, wherein moving the tines to a spaced apart configuration comprises deforming the resilient body.

20. A method according to claim 18, wherein moving a portion of a mandrel into the body opening comprises urging a flared portion of the mandrel against the resilient body or at least one of the tines.

21. A method according to claim 18, wherein urging at least a portion of the tines toward one another comprises permitting elastic recovery of the resilient body.

22. A method according to claim 21, wherein permitting elastically recovery of the resilient body comprises removing a deforming stress.

23. A method according to claim 22, wherein removing the deforming stress comprised at least partially withdrawing the mandrel from the opening.

24. A method according to claim 18, wherein engaging tissue comprises at least partially piercing the tines into the tissue.

25. A method according to claim 18, further comprising approximating a pledget portion of the staple to the wound.

26. A method according to claim 18, wherein engaging tissue comprises using a pusher to urge the staple towards the tissue.
